(19) United States
(12) Patent Application Publication
(10) Pub. No.: US 2009/0234298 A1
(43) Pub. Date: Sep. 17, 2009

(54) INJECTION DEVICE

(76) Inventors: Rosemary Louise Habeshaw, Cambridge (GB); Johathan Hogwood, Hertfordshire (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/910,546

(22) PCT Filed: Mar. 21, 2006
(86) PCT No.: PCT/GB2006/001023

§ 371 Date: Nov. 11, 2008

(30) Foreign Application Priority Data
Apr. 6, 2005 (GB) 0507015.6

(51) Int. Cl. A61M 5/31

(52) U.S. Cl. 604/195

(57) ABSTRACT

An injection device (110) comprises a housing (112) defining a first axis (101). A drive (120) acts upon a syringe when released by a trigger (114). The trigger is rotatable from a rest position in which the drive is retained to an active position in which it no longer causes the drive to be so retained. The trigger is pivotally mounted and has a surface (201) shaped such that a user can apply a force in a direction substantially parallel to the first axis to rotate the trigger from its rest position to its active position. Such an injection device provides improved handling and ease of operation.

### FIELD OF THE INVENTION

The present invention relates to an injection device of the type that receives a syringe, extends it, discharges its contents and then retracts it automatically.

### BACKGROUND OF THE INVENTION

Previously known injection devices are shown in WO 95135126 and EP-A-0 516 473 and tend to employ a drive spring and a trigger that, when activated, causes the drive spring to act on the syringe when a releasable locking mechanism is also engaged.

The trigger acts is rotatable about an axis so that when it is depressed at a first end, a second end (which normally engages with the drive spring) is also rotated, thereby releasing the drive spring, extending the syringe and discharging its contents. The trigger is generally mounted in the side of the housing of the injection device parallel to the longitudinal axis of the body of the injection device. The trigger is rotated by pressing down on the trigger at one end in a direction towards the housing.

A problem with such devices is that the action of pressing down on the trigger towards the housing can be difficult if the device is being held in one hand. This poses significant problems for sick and ill users of the injection device, for example users suffering from rheumatoid arthritis.

### SUMMARY OF THE INVENTION

The injection device of the present invention is designed to deal with this and other problems.

In view of the foregoing and in accordance with a first aspect of the invention, there is provided an injection device comprising:
a housing defining a first axis, and being adapted to receive a syringe having a discharge nozzle, so that the syringe is movable between a retracted position in which the discharge nozzle is contained within the housing and an extended position in which the discharge nozzle extends from the housing through an exit aperture;
a drive that is acted upon and in turn acts upon the syringe; and
a trigger rotatable from a rest position, in which it causes the drive to be retained, to an active position, in which it no longer causes the drive to be so retained, thus allowing the contents of the syringe to be discharged through the discharge nozzle,
wherein the trigger is pivotally mounted and has a surface shaped such that a user can apply a force in a direction substantially parallel to the first axis to rotate the trigger from its rest position to its active position.

By having the trigger operable so that a force can be applied in a direction which is substantially parallel to the first axis, the trigger does not need to be pushed into the side of the housing to activate it. This way, the injection device can be held and operated with one hand by sliding the hand down the housing and over the trigger as the exit aperture is pushed against a user's body.

Preferably, the surface is a first concave surface.

In one embodiment of the invention, the surface is provided with a plurality of ridges.

Preferably, the housing is provided with a second concave surface substantially opposite to the trigger so that a user can grip the device and apply a force to the trigger and second concave surface in a direction substantially parallel to the first axis.

In one embodiment of the invention, the housing comprises an abutment located adjacent the trigger between the trigger and the exit aperture.

In one embodiment of the invention, the second concave surface is provided with a plurality of ridges.

### BRIEF DESCRIPTION OF THE DRAWINGS

The invention will now be described by way of example with reference to the accompanying drawings, in which:

FIG. 1 shows a perspective view of an injection device according to the present invention;

FIG. 2 shows an alternative perspective view of an injection device according to the present invention; and

FIG. 3 shows a side view of the injection device of FIGS. 1 and 2 with an upper section of the housing of the injection device removed.

### DETAILED DESCRIPTION OF THE DRAWINGS

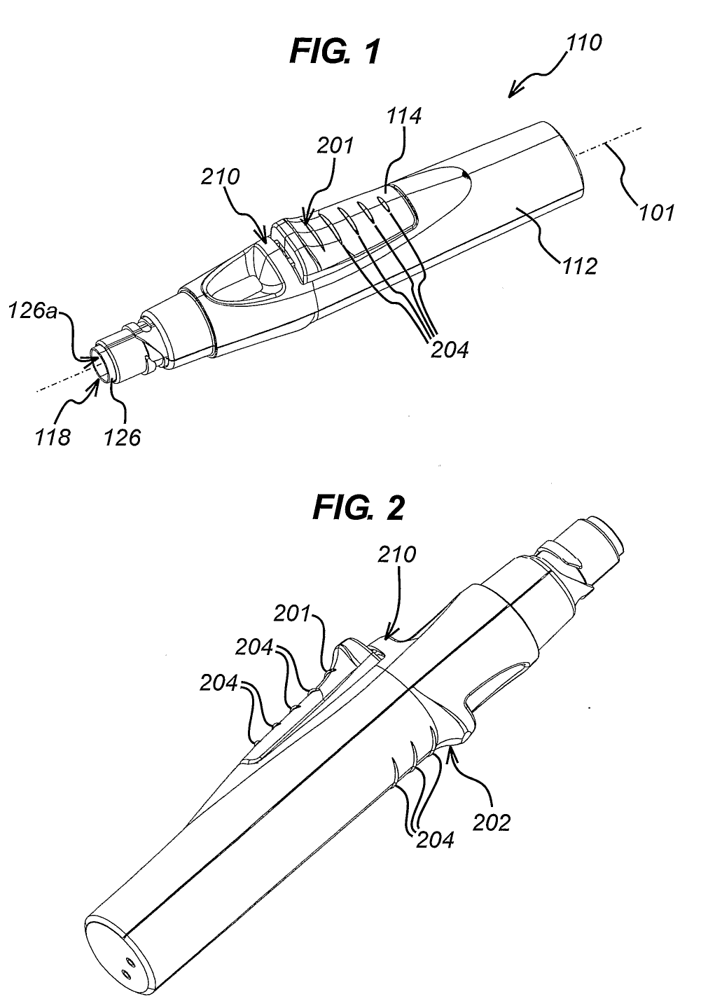

FIGS. 1 to 3 show an injection device 110 according to a first embodiment of the present invention. The injection device 110 has an injection device housing 112 and a longitudinal axis 101.

A syringe (not shown) is contained in the housing 112. The injection device 110 comprises trigger 114 and a releasable locking mechanism 116. The trigger 114 has a first end 114a and a second end 114b. The trigger 114 is rotatable about a pivot 115 from a rest position to an active position. The second end 114b of the trigger 114 connects with a drive coupling 121 which is acted upon by a drive spring 120. The drive coupling 121 is in communication with the syringe.

Rotation of the trigger 114 about the pivot 115 in a direction R (i.e. downwards into the housing 112 at its first end 114a) causes the second end 114b of the trigger 114 to disengage from the drive coupling 121, thereby letting the drive spring 120 drive the syringe (via the drive coupling 121) along the longitudinal axis 101 and out of an aperture 118 in the housing 112.

The releasable locking mechanism 116 is in communication with sliding sleeve 126 which protrudes, when in a first position, from the aperture 118 in the housing 112. The locking mechanism 116 is deactivated by movement of the sliding sleeve 126 along the longitudinal axis 101 into the housing 112 into a second position.

A first end 126a of the sliding sleeve 126 can be placed against a body into which drug is being delivered, thereby deactivating the releasable locking mechanism 116 and allowing the trigger 114 to rotate in direction R from its rest position to its active position.

The trigger 114 is shaped in such a way that there is a section of surface 201 at an angle to the longitudinal axis 101 of the injection device 110. Hence, rotation of the trigger 114 can take place by exertion of force in a direction which is not necessarily perpendicular to the longitudinal axis. This way, a rotational force can still be exerted on the trigger 114, even if that force is not applied inwards towards the housing 112.

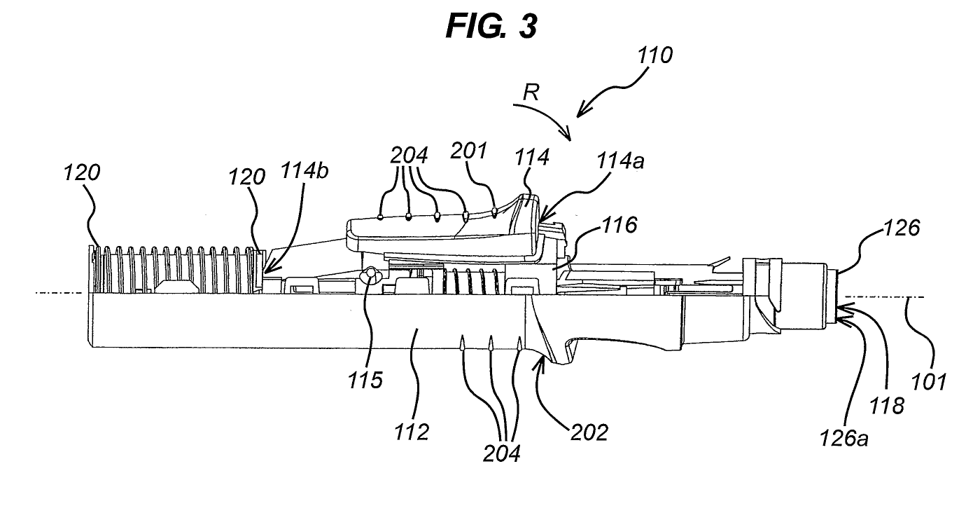

In the embodiment of the invention shown in FIGS. 1 and 3, the surface 201 is shaped concavely.

The hand of a user of the injection device 110 can be wrapped around the housing 112 and the injection device 110 forced against the user's body so that the sliding sleeve 126 causes the locking mechanism 116 to disengage. The trigger 114 can now be activated by movement of the user's hand across the housing 112 and over the trigger 114 so that a force having a component in the direction of the longitudinal axis 101 is applied to the trigger 114 causing it to rotate.

The housing 112 also comprises a ridge 210 (or an abutment) located behind the trigger 114 towards the exit aperture 118 of the housing 112. The ridge acts as a stop surface to prevent a user's hand moving further down the housing 112 once the trigger 114 has been activated. This way, the injection device 110 can be held firmly against a user's body after activation of the trigger 114 and whilst the contents of the syringe is being dispelled into their body.

The housing 112 comprises a second concave surface 202 located substantially opposite to the trigger 114. Thus, the user's hand is prevented from moving further down the housing 112 once the trigger 114 has been activated.

The surface 201 and a section of the surface of the housing opposite the trigger 114 both comprise ridges 204 which act as grips for the user's hand.

It will of course be understood that the present invention has been described above purely by way of example and modifications of detail can be made within the scope of the invention.

1. An injection device comprising:
a housing defining a first axis, and being adapted to receive a syringe having a discharge nozzle, so that the syringe is movable between a retracted position in which the discharge nozzle is contained within the housing and an extended position in which the discharge nozzle extends from the housing through an exit aperture;
a drive that is acted upon and in turn acts upon the syringe; and
a trigger rotatable from a rest position, in which it causes the drive to be retained, to an active position, in which it no longer causes the drive to be so retained, thus allowing the contents of the syringe to be discharged through the discharge nozzle,
wherein the trigger is pivotally mounted and has a surface shaped such that a user can apply a force in a direction substantially parallel to the first axis to rotate the trigger from its rest position to its active position.

2. The injection device of claim 1, wherein the surface is a first concave surface.

3. The injection device of claim 1 or claim 2, wherein the surface is provided with a plurality of ridges.

4. The injection device of anyone of the preceding claims, wherein the housing is provided with a second concave surface substantially opposite to the trigger so that a user can grip the device and apply a force to the trigger and second concave surface in a direction substantially parallel to the first axis.

5. The injection device of claim 4, wherein the second concave surface is provided with a plurality of ridges.

6. The injection device of any one of the preceding claims, wherein a section of the surface of the housing substantially opposite to the trigger includes a plurality of ridges.

7. The injection device of any one of the preceding claims wherein the housing comprises an abutment located adjacent the trigger between the trigger and the exit aperture.

8. An injection device substantially as hereinbefore described with reference to and as shown in the attached drawings.
